(19) United States
(12) Patent Application Publication
(10) Pub. No.: US 2023/0096558 A1
(43) Pub. Date: Mar. 30, 2023

(54) CELL SEPARATION PARTICLES FOR AND/NOT OPERATIONS OR MULTIPLE TARGETS

(71) Applicant: Charles Stark Draper Laboratory, Inc., Cambridge, MA (US)

(72) Inventor: Jason O Fiering, Boston, MA (US)

(73) Assignee: Charles Stark Draper Laboratory, Inc., Cambridge, MA (US)

(21) Appl. No.: 17/955,738

(22) Filed: Sep. 29, 2022

Related U.S. Application Data
(60) Provisional application No. 63/250,156, filed on Sep. 29, 2021.

(51) Int. Cl. G01N 1/40; C12N 13/00; B01D 21/28

(52) U.S. Cl. CPC G01N 1/4077; C12N 13/00; B01D 21/283; G01N 2001/4094

(57) ABSTRACT

A method of acoustophoresis using selection particles that alter acoustic response is provided. The method can include selecting a set of selection particles based on surface markers of a plurality of target particles to be separated using acoustophoresis. The method can include incubating the set of selection particles with the plurality of target particles in a solution such that the set of selection particles bind with the surface markers on the plurality of target particles to create a plurality of bound particles. The method can include providing the plurality of bound particles to an acoustophoresis device tuned to separate the particles based on a net acoustic contrast between each of the plurality of bound particles. The method can include receiving a plurality of output streams from the acoustophoresis device that each include a respective bound particle of the plurality of bound particles.

### CROSS-REFERENCE TO RELATED APPLICATIONS

This application claims the benefit of and priority to U.S. Provisional Patent Application No. 63/250156, filed on Sep. 29, 2021, the contents which is incorporated by reference herein in its entirety for all purposes.

### BACKGROUND

Cell isolation procedures often rely on surface markers, for example, through antibody labeling. However, antibody labeling and corresponding cell sorting techniques suffer from poor yield and very low throughput. Other techniques also suffer from poor sensitivity to surface markers.

### SUMMARY

Applications in cell therapy, diagnostics, and research often require isolation of cell types according to their surface markers (e.g., surface proteins). This can be achieved by antibody labeling along with flow cytometry sorting (e.g., fluorescence assisted cell sorting (FACS), etc.) or by attaching magnetic micro or nano-particles to the cells and trapping them with a magnet (e.g., magnet assisted cell sorting (MACS), etc.). However, FACS is costly, slow, and suffers from poor yield of selected cells. Likewise, magnetic selection is generally binary and has poor sensitivity to valences of surface markers or combinations of surface markers.

The systems and methods of this technical solution improve upon other inferior approaches by taking advantage of high-throughput of acoustophoresis while allowing selection of cells by combinations of surface markers in a way that other approaches, such as FACS and MACS, cannot achieve. The systems and methods described herein utilize microfluidic cell separation by acoustophoresis, where cells are pre-treated by binding them with customized particles to alter their response to the acoustic field when processed through the separation device. The customized particles alter the acoustic contrast of the cells, or other target particles to which the customized particles are bound, and thereby alter their trajectory through the acoustic separator. The customized particles can enable discrimination of cell or target particle types that would otherwise could not be separated from one another in an untreated (sometimes referred to as “label-free”) state.

At least one aspect of the present disclosure is directed to a method of acoustophoresis using selection particles that alter acoustic response of particles in suspension. The method can include selecting a plurality of selection particles based on a first marker of a first target particle and a second marker of a second target particle. The plurality of selection particles can include a first selection particle having a greater acoustic contrast and a second selection particle having a lesser acoustic contrast. The method can include incubating the plurality of selection particles in a solution comprising at least the first target particle and the second target particle, such that the first selection particle binds to the first marker of the first target particle and the second selection particle binds to the second marker of the second target particle. The method can include providing the solution to an acoustophoresis channel configured to separate the first target particle from the second target particle. The method can include receiving a first output stream comprising the first target particle bound to the first selection particle and a second output stream comprising the second target particle bound to the second selection particle from the acoustophoresis channel.

In some implementations, the first target particle and the second target particle comprise cells. In some implementations, the first marker and the second marker are expressed on a surface of a cell membrane of each of the first target particle and the second target particle, respectively. In some implementations, the first marker and the second marker comprise a protein. In some implementations, the solution comprises three or more target particles, the three or more target particles including the first target particle and the second target particle. In some implementations, the solution is provided to the acoustophoresis channel after the plurality of selection particles have incubated in the solution for a predetermined amount of time.

In some implementations, the method can include controlling a transducer connected to the acoustophoresis channel to carry out acoustophoresis. In some implementations, the method can include providing a fluid additive having predetermined density and compressibility with the solution to the acoustophoresis channel to modify an acoustic contrast of the first target particle and the second target particle in the solution including the fluid additive. In some implementations, providing the solution to the acoustophoresis channel comprises flowing the solution through the acoustophoresis channel. In some implementations, the acoustophoresis channel comprises a first outlet that provides the first output stream and a second outlet that provides the second output stream.

In some implementations, the solution further comprises a third particle, and the first output stream received from the acoustophoresis channel comprises the third particle. In some implementations, the method can include providing the first output stream and a fluid additive to a second acoustophoresis channel configured to separate the first target particle from the third particle based on a net acoustic contrast of a complex of the first target particle and the first selection particle and an acoustic contrast of the third particle, in the second acoustophoresis channel. In some implementations, the method can include receiving a third output stream comprising the first target particle and a fourth output stream comprising the third particle.

At least one other aspect of the present disclosure is directed to another method of acoustophoresis using selection particles that have predetermined size, density, and compressibility. The method can include selecting a first selection particle and a second selection particle based on a first marker and a second marker of a first target particle and based on a second target particle, the first selection particle having a high acoustic contrast when suspended in a selected fluid. The method can include incubating the first selection particle and the second selection particle in a solution comprising the selected fluid. The selected fluid can include the first target particle and the second target particle, such that the first selection particle binds to the first marker of the first target particle and the second selection particle binds to the second marker of the first target particle. The method can include providing the solution to an acoustophoresis channel configured to separate the first target particle from the second target particle based on a difference between a net acoustic contrast of a complex of the first selection particle, the second selection particle, and the first target particle and an acoustic contrast of the second target particle. The method can include receiving a first output stream comprising the first target particle bound to the first selection particle and the second selection particle and a second output stream comprising the second target particle from the acoustophoresis channel.

In some implementations, the solution is provided to the acoustophoresis channel after the first and second selection particles have incubated in the solution for a predetermined amount of time. In some implementations, the method can include providing a fluid additive having a predetermined density and compressibility with the solution to the acoustophoresis channel to modify an acoustic contrast of the complex of the first target particle bound to the first selection particle and the second selection particle, and an acoustic contrast of the second target particle. In some implementations, the method can include controlling a transducer that causes the first target particle bound to the first selection particle and the second selection particle to be forced towards a center of the acoustophoresis channel.

One other aspect of the present disclosure is directed to a system. The system can include an acoustophoresis device comprising a microfluidic channel. The acoustophoresis device can receive a solution including a first target particle, a second target particle, a first selection particle, and a second selection particle. The solution can have been incubated such that the first target particle is bound to the first selection particle and the second selection particle in the solution such that a net acoustic contrast of a complex of the first target particle, the first selection particle, and the second selection particle differs from an acoustic contrast of the second target particle. The acoustophoresis device can perform acoustophoresis in the microfluidic channel. The acoustophoresis device can provide a first output stream comprising the first target particle bound to the first selection particle and the second selection particle, and a second output stream comprising the second target particle.

In some implementations, the system can include a container comprising incubation media including the first selection particle, the second selection particle, the first target particle, and the second target particle, such that the first selection particle and the second selection particle bind to the first target particle in the incubation media. In some implementations, the container is fluidly coupled to an inlet of the microfluidic channel of the acoustophoresis device. In some implementations, the incubation media is provided at least as part of the solution to the microfluidic channel of the acoustophoresis device. In some implementations, the microfluidic channel is a first microfluidic channel. In some implementations, the acoustophoresis device further comprises a second microfluidic channel that receives one of the first output stream or the second output stream and performs acoustophoresis.

In some implementations, the method can include controlling a transducer coupled to the acoustophoresis channel to carry out acoustophoresis. In some implementations, the method can include providing at least one of the first output stream or the second output stream as input to a second acoustophoresis channel. In some implementations, the first selection particle and the third selection particle comprise the same materials.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The foregoing and other objects, aspects, features, and advantages of the disclosure will become more apparent and better understood by referring to the following description taken in conjunction with the accompanying drawings, in which:

FIGS. 1A and 1B illustrate types of low and high contrast particles, as described herein, as well as cells bound to those particles to provide both low and high acoustic contrast when suspended in a fluid, in accordance with one or more implementations;

FIG. 2 illustrates a diagram of an example multi-stage acoustophoresis separation system operating on cells bound to high and low contrast particles, in accordance with one or more implementations;

FIG. 3 illustrates a diagram of another example multi-stage acoustophoresis separation system used to isolate target cell types, in accordance with one or more implementations; and

FIG. 4 illustrates a flow diagram of an example method of selective separation of target cells using selection particles in acoustophoresis, in accordance with one or more implementations.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes systems and methods for separation devices that leverage selection of target particles through combinations of surface markers that alter their acoustic contrast. In particular, the systems and methods described herein provide high-throughput acoustophoresis separation devices that can operate to separate target particles. By using multi-stage configurations of acoustophoresis devices, the systems and methods described herein can be utilized to perform binary separation or selection operations across multiple target particles.

In other words, the systems and methods described herein utilize more than one particle type, along with predetermined modulation of the net acoustic contrast of the system, to achieve highly specific separation. For example, if a desired cell or target particle must have surface marker A and B, only those cells or target particles with the A and B markers will be selected, while those that have only one of A or B will not be selected. Likewise, Boolean “NOT” conditions can be achieved by selecting for cells that have A or B but not both. In addition, multiple stage selections are possible from a single treatment, whereby a first subpopulation of cells or target particles are selected, and then that population is further fractionated in a second stage with a modified suspending medium.

In some implementations, the acoustic contrast of the selection particles can be a result of their manufacture, and can reflect the material of their composition, their porosity, and any “shell,” or other feature. Some examples of such selection particles can include, but are not limited to, gas-filled particles, oil-filled particles, or porous particles, such that they have low density and high compressibility. By having such properties, these selection particles can result in low acoustic contrast (e.g., relative to a separation medium or fluid flow, etc.). In some implementations, some example particles can include high-density materials, such as nano-particles of gold, iron-oxide, or other high-density materials. Such selection particles can have a high density and low compressibility, resulting in high acoustic contrast (e.g., relative to a separation medium or fluid flow, etc.).

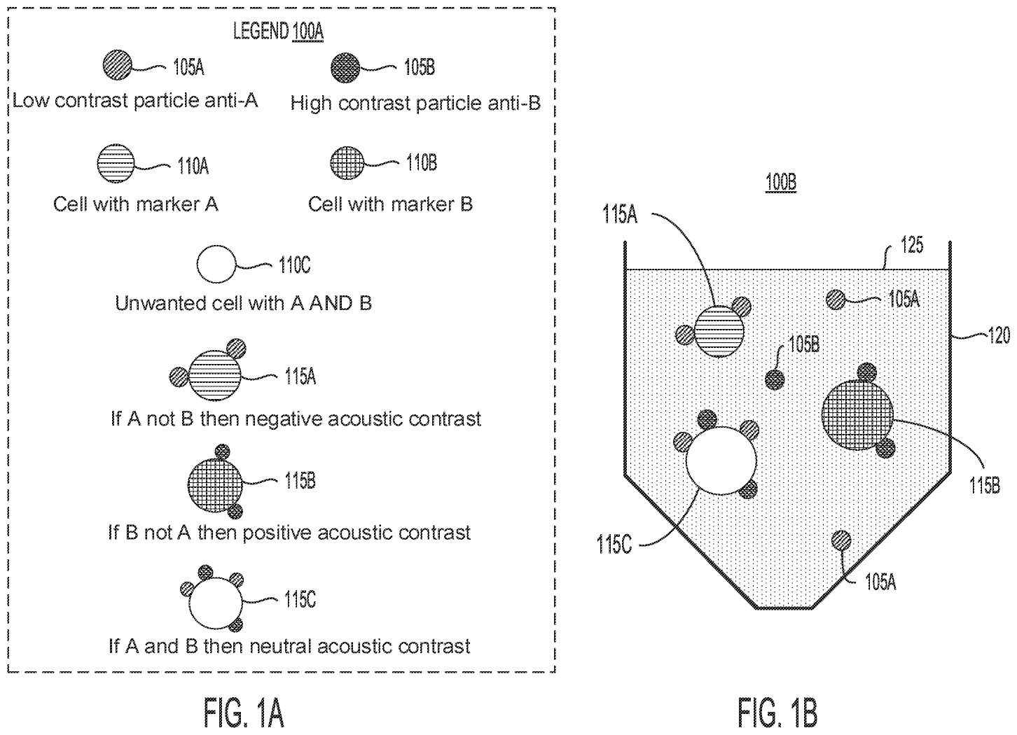

By using combinations of particles with different characteristics (e.g., relative acoustic contrast to a separation medium used in acoustophoresis, etc.), the acoustic separation process can be manipulated to achieve specific cell or target particle selection. Examples of these are provided in FIGS. 1A, 1B, 2, and 3. As described herein, the terms “low acoustic contrast” and “high acoustic contrast” should be understood to correspond to the relative acoustic contrast between the target particles (and any selection particles bound thereto) and the separation medium in which the target particles are suspended. In some implementations, undesired (e.g., waste) particles may be bound to specific selection particles, and a separation medium can be specifically chosen to create an acoustic contrast appropriate to separate the waste particles from the target particles (e.g., cells, etc.).

The acoustic contrast (sometimes referred to as an “acoustic contrast factor”) of a particle in a solution is a result of the relationship between the density and compressibility of the fluid and that of the particle when suspended in the solution. Particles with high acoustic contrast can correspond to particles having an acoustic contrast factor that is greater than a predetermined threshold (e.g., 0.0, 0.01, 0.05, etc.). Particles with low acoustic contrast can correspond to particles having an acoustic contrast factor that is less than a predetermined threshold (e.g., 0.0, −0.01, −0.05, etc.). The relative acoustic contrast of particles suspended in the solution may depend on the density and the sound velocity of the solution. Density media or other fluids can be added to solutions as described herein to modify the relative acoustic contrast of the particles suspended therein.

Referring now to FIG. 1A, shown is an example legend 100A showing various particles described herein. As shown in the legend 100A, at least two selection particles 105A and 105B are present. The selection particle 105A is designated as “anti-A,” indicating that it can bind to cells or other target particles with a “marker A.” As described herein, the term “binding,” in the context of binding a selection particle to a target particle, can refer to a variety of bonding techniques or affinity molecules. For example, selection particles may bind to target particles using any of antibodies, aptamers, antigen-binding fragments, lectins, or other biochemistry-related molecules that allow a selection particle to bind to a surface or portion of a target particle.

The term “marker” as described herein can be any type of cellular marker or target particle marker, which may be any surface feature, molecule, protein, antibody, or other chemical or physical feature. For example, a marker can be an antibody marker or a protein present on the external cell membrane of a cell. In general, a marker can be any type of surface feature to which other particles may bind. In some implementations, such cell membranes can include multiple types of cell markers. In FIG. 1A, the selection particle 105A is indicated as a “low contrast particle.” Low contrast particles are particles with a low acoustic contrast relative to typical separation media used in acoustophoresis. As described herein low contrast particles can include gas-filled particles, oil-filled particles, or porous particles, such that they have low density and high compressibility.

The selection particle 105A is designated as “anti-A,” indicating that it can bind to cells or other target particles with a “marker A.” Such markers can be any type of cellular marker or target particle marker. For example, a marker can be an antibody marker or a protein present on the external cell membrane of a cell. In some implementations, such cell membranes can include multiple types of cell markers. The selection particle 105A is indicated as a “low contrast particle.” Low contrast particles are particles with a low acoustic impedance relative to typical separation media used in acoustophoresis. As described herein, high-contrast selection particles can have a high density and low compressibility, resulting in high acoustic contrast (e.g., high impedance relative to a separation medium.). When the low-contrast selection particles 105A or the high-contrast selection particles 105B bind to a target particle (e.g., bind on one or more markers at the cell or target particle) the selection particles 105A or 105B can effect a composite acoustic contrast that is different from that of the acoustic contrast of the target particles or cells themselves. Although only two selection particles 105A and 105B are shown in the legend 100A, it should be understood that any number and variety of selection particles can be used to achieve a desired outcome.

As described herein, the selection particles 105A and 105B can bind to corresponding markers on cells or other target particles. Certain markers can be expressed by certain target cells, or can be present on the surface of other target particles. The selection particles 105A can be placed in a solution with the target cells or target particles, and can subsequently bind to appropriate markers expressed on the surface of the target cells. As shown in legend 100A, three example target cells are shown: a target cell 110A having marker “A,” a target cell 110B having marker “B,” and an unwanted cell having both markers A and B. It should be understood that the marker designations “A” and “B” are used purely as placeholder examples for simplicity and brevity, and in reality can be any type of marker (e.g., protein, antibody binding site, etc.) expressed on the surface of a cell (e.g., a cell surface marker). In some implementations, for target particles that are not cells, a marker can be, for example, a small feature (e.g., nano-particle, partial surface coating, etc.) that allows any of the selection particles 105A or 105B to bind to the target particle.

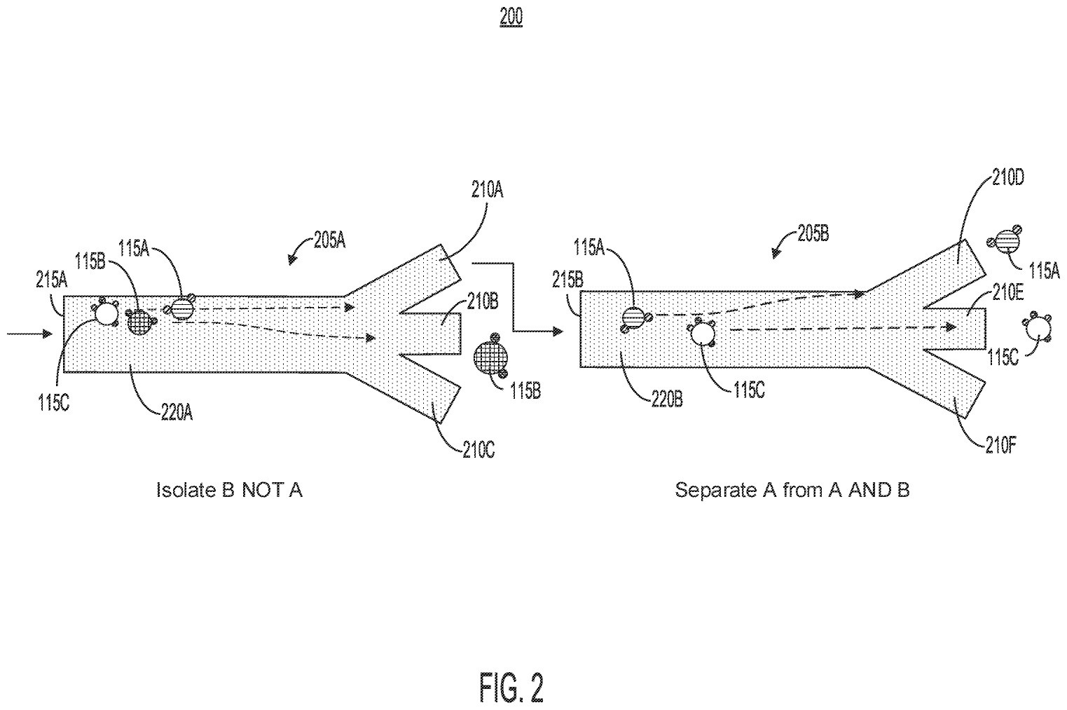

In the first example shown through FIGS. 1A, 1B, and 2, three target cells are shown, but it should be understood that any number of types of target particles can bind to any number of selection particles, and that three target cells are shown in legend 100A for simplicity. The target cell 110A, as indicated, has the marker A expressed on its surface and does not have the marker B expressed on its surface. Therefore, when the target cell 110A is exposed selection particles 105A and 105B, it is likely to bind to the selection particle 105A and not the selection particle 105B. In contrast, the target cell 110B, as indicated, has the marker B express on its surface and does not have the marker A expressed on its surface. Therefore, when the target cell 110B is exposed selection particles 105A and 105B, it is likely to bind to the selection particle 105B and not the selection particle 105A. In addition to target cells, the selection particles 105A and 105B may be carefully chosen or manufactured to correspond to an unwanted or waste particle, which can be any type of cell or target particle that is to be separated from desired or target cells or target particles.

The legend 100A further shows the selection particles 105A and 105B bound to the target cells 110A and 110B, and the undesired cell 110C. Collectively, these are referred to herein as the bound cells 115A, 115B, and 115C, corresponding respectively to the target cells 110A and 110B, and the undesired cell 110C. As shown, because the target cell 110A includes the “A” marker expressed on its cell surface, and does not include the “B” marker, the low-contrast selection particle 105A has bound to its surface, resulting in the bound cell 115A, which has negative acoustic contrast relative to its previous state as the unbound target cell 105A. The target cell 110A lacks the “B” marker, and thus the selection particle 110B does not bind to the surface of the target cell 110A. Likewise, because the target cell 110B includes the “B” marker expressed on its cell surface, and does not include the “A” marker, the high-contrast selection particle 105B has bound to its surface, resulting in the bound cell 115A, which has negative acoustic contrast relative to its previous state as the unbound target cell 105A. The target cell 110B lacks the “C” marker, and thus the selection particle 110B does not bind to the surface of the target cell 110A.

Certain target cells or target particles may bind to more than one type of selection particle. As shown in the legend 100A, the unwanted cell 110C (e.g., a waste cell in this example, etc.) includes both the A and the B markers. Therefore, as shown, the corresponding bound cell 115C binds to both the low-contrast selection particle 105A and the high-contrast selection particle 105B. This results in a net neutral acoustic contrast for the bound particle 115C, and will therefore respond differently than the bound particles 115A and 115B when exposed to acoustophoresis. Likewise, each of the bound particles 115A and 115B will behave differently when exposed to acoustophoresis. By using an acoustophoresis device that is manufactured to leverage these differences, precise and high-throughput cell separation can be achieved.

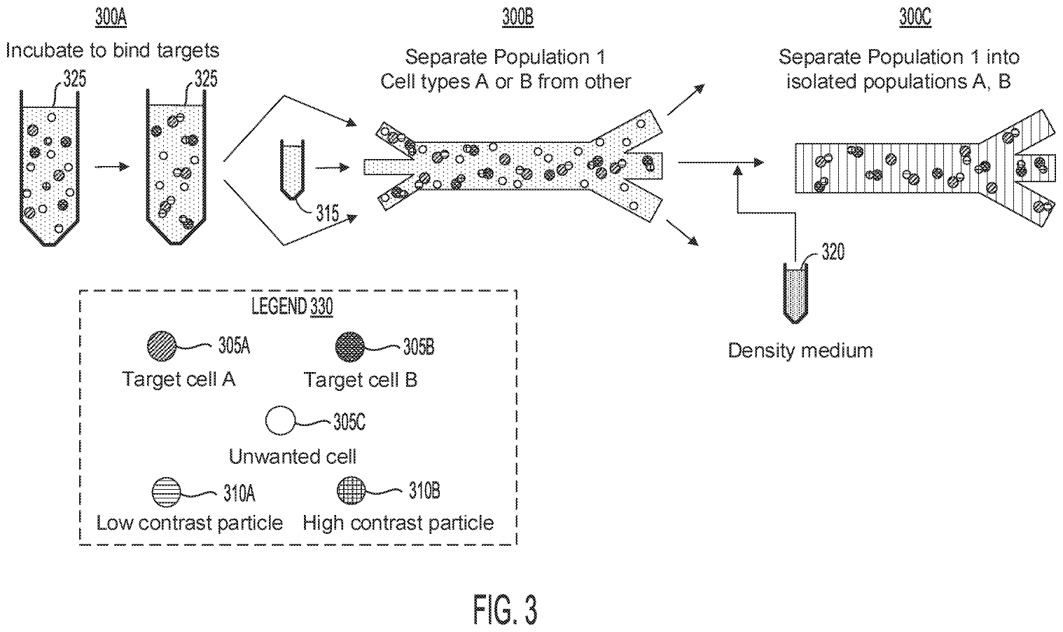

Referring briefly now to FIG. 1B, illustrated is an example cross-sectional diagram 100B of an incubation process in which the target cells 110A and 110B, and the undesired cell 110C, are bound to the low-contrast selection particles 105A and the high-contrast selection particles 105B. As shown in the diagram 100B, target cells 110A and 110B, and the undesired cell 110C, are suspended in an incubation media 125, which may be contained in a container 120. The container 120 can be any type of container suitable for storing the incubation media 125 and the various particles described herein. The container 120 may serve as part of a system that includes an acoustophoresis device, such as the devices described in connection with FIGS. 2 and 3. The incubation media 125 can be any type of media capable of supporting the target cells 110A and 110B, the undesired cell 110C, and the selection particulars 105A and 105B.

It should be understood that the particular configuration shown in the diagram 100B is an example implementation of the present technology, and should not be considered as limiting to the types of the incubation media 125 or environments to which selection particles may be bound to target cells or target particles, as described herein. As shown, the selection particles 105A have bound to the target cells 110A, resulting in the bound cells 115A as described herein. Likewise, the selection particles 105B have bound to the target cells 110B, resulting in the bound cells 115B as described herein. The selection particles 105A and 105B have also both bound to the undesired cell 110C, which results in the bound cell 115C, having neutral acoustic contrast. An example of these cells being applied to an acoustophoresis process is shown in FIG. 2.

Referring briefly now to FIG. 2, illustrated is a top-view block diagram 200 of an example multi-stage acoustophoresis separation device operating on cells bound to high and low contrast particles, in accordance with one or more implementations. As shown, the diagram 200 includes a first acoustophoresis stage 205A having an inlet 215A, a central channel 220A, and multiple outlet channels 210A, 210B, and 210C. An acoustophoresis stage 205A can be a microfluidic channel that can receive an acoustic separation media (e.g., which may be, or may be similar to, the incubation media 125 of FIG. 1B) including any of the cells 110A, 110B, and 110C, or the bound particles 115A, 115B, and 115C. Using the separation techniques described herein (e.g., aided by the selection particles, etc.), one or more of the bound particles 115A, 115B, and 115C can be separated from the other cells in the media. The central channel 220A can be a microchannel fabricated from a substrate, such as silicon, glass or quartz, or a polymer with high acoustic impedance, such as polystyrene. . The microchannel can be fabricated using any suitable technique, including but not limited to molding (e.g., injection molding), etching, embossing, laser ablation, or combinations thereof. The central channel 220A can be rectangular in cross section, with width and height dimensions that can range from 100 μm to 1000 μm. However, it should be understood that other sizes are possible to achieve a desired outcome, including, for example, 0 μm to 5 μm, 10 μm to 100 μm, or 1000 μm to 10000 μm. The central channel 220A need not necessarily be rectangular in cross-section, and may include any type of suitable geometry, such as a curved geometry, an elliptical geometry, a circular geometry, a hexagonal geometry, or an octagonal geometry, among others.

The inlet 215A of the first stage 205A can receive a suspension media including target cells or target particles (e.g., which may be bound to the various selection particles described herein). The suspension media can be, for example, saline, or another suitable suspension media that will not interfere with the cells or target particles. The inlet 215A of the first stage 205A can be an inlet port that can be connected to a pipe, a tube, a reservoir, a pump, or another microfluidic feature that provides the suspension media including the target cells or target particles. In some implementations, the first stage 205A can receive the flow of suspension media from another stage in a microfluidic system. In some implementations, the first stage 205A may be one of many parallel acoustophoresis channels, for example, to form a system that provides increased throughput of separated cells, which themselves may be defined on one or more layers of a microfluidic system. As shown, the inlet 215A can be in fluid communication with the central channel 220A of the first stage 205A.

The outlets 210A, 210B, and 210C of the first stage 205A can provide streams of the suspension media including target cells or target particles (e.g., which may be bound to one or more of the various selection particles described herein) following an acoustophoresis process. In general, each of the outlets can align (e.g., along the length of the acoustophoresis channel) with a respective pressure node or pressure anti-node induced by a transducer acting on the microchannel during the operation of the first acoustophoresis stage 205A. The outlets 210A, 210B, and 210C of the first stage 205A can be outlet ports, which can be connected to (e.g., via any type of connector or fastener, etc.) a pipe, a tube, a reservoir, a pump, or another microfluidic feature that can receive an output flow of the separated suspension fluid. Although three outlets 210A, 210B, and 210C are shown, it should be understood that any number of outlets may be implemented using the techniques described herein, for example, such that each outlet aligns with a respective pressure node or anti-node induced in the suspension media by a transducer. In some implementations, any one of the outlets an acoustophoresis device can be connected (e.g., via one or more connectors, tubes, pipes, etc.) to other microfluidic processing stages or other microfluidic features, as described herein.

To achieve acoustophoresis, a wall of the microfluidic channel in the first acoustophoresis stage 205A can be coupled to an ultrasonic oscillator, such as a piezoelectric transducer. The transducer can be electrically driven to excite the channel such that some of the cells 110A, 110B, or 110C migrate toward the axial center stream of the channel as they flow through it. In this example, in stage 205A, the relative acoustic contrast of the bound cell 115B causes the bound cell 115B to migrate toward the axial center stream of the channel, and exit the acoustophoresis stage 205A via the center outlet 210A. Once separated, the bound cells 115B can be provided to other microfluidic devices, stored in a reservoir, or otherwise subjected to any type of post-separation process. As shown, the relatively lower acoustic contrast of the bound cells 115A and 115C cause the bound cells to migrate toward the side outlet 210A, which in this example flows into the inlet of the second acoustophoresis stage 205B. However, it should be understood that arrangements are also contemplated, for example, where microfluidic systems can have any number of stages, inlets, outlets, or central channels, and where any number of outlets of one or more stages can be fluidly coupled to one or more inlets of further microfluidic stages, systems, or devices, to achieve desired results.

The migration rate of the cells can depend on their size, density, and compressibility relative to the surrounding media, and therefore differences in the acoustic contrast of the cells can be such that some cell types (e.g., the bound cell 115B, etc.) will migrate toward the center outlet 210B, while others (e.g., the bound cells 115A and 115B) will be directed to the side outlet 210A. An example derivation of the effect of selection particles (e.g., selection particles 105A and 105B, etc.) on the target cells (e.g., the target cells 110A and 110B, etc.) is provided as follows. The response of a cell or particle (e.g., such as the target cells 110A or 110B, or the undesired cell 110C, etc.) to manipulation by acoustophoresis is commonly predicted to behave according to its size and its acoustic contrast. For example, in an example configuration in a resonant cavity the Force, F, on the particle is written as:

F˜ΦV

The relationship above emphasizes that the force F on the particle is proportional to 4:13 (the acoustic contrast) and V (the particle volume). The magnitude of the force can depend on properties of the system, such as the frequency or energy applied to the acoustophoresis device, and the position that the force is applied within the resonant cavity, but these properties can be held constant to account for these differences. The acoustic contrast itself can be calculated from the particle's density and compressibility, and from the density and compressibility of the suspending fluid (e.g., the media in which the bound cells 115A, 115B, and 115C are suspended).

In the techniques described herein, the selection particles (e.g., the selection particles 105A and 105B, etc.) are used to alter the net contrast on the complex of a target cell or target particle when the selection particle(s) bind to the target cell and the target particle. The behavior of an aggregate or complex of multiple cells and particles can be calculated based on the assumption that the effective contrast of the complex (e.g., the selection particle bound to the cell or target particle) is an average of each component's contrast weighted by its volume:

$${Φ}^{′} = \frac{{Φ}_{1} {V}_{1} + {Φ}_{2} {V}_{2} + {Φ}_{3} {V}_{3}}{{V}_{1} + {V}_{2} + {V}_{3}} ,$$

and the force on the aggregate would be estimated to vary as:

F˜Φ₁V₁+Φ₂V₂+Φ₃V₃ . . .

The subscripts in the relationships above indicate each individual particle in the aggregate. The sum of the contributions can be termed the acoustophoretic mobility, with units of volume. Some example values of particle size and contrast factor are given in Table 1 below:

TABLE 1
Particle Size and Acoustic Contrast Factor

| | Typical | Approximate Contrast |
|---|---|---|
| Cell/Particle | Diameter (μm) | Factor in Saline fluid |
| Red blood cell | 7 | 0.16 |
| T lymphocyte | 7 | 0.10 |
| Polystyrene microbead | selectable | 0.39 |
| Silicone oil emulsion droplet | selectable | −0.8 |
| Gas bubble | selectable | −2 |

Therefore, as an example, in a saline solution (e.g., a suspension media) a bound complex of a T-cell (e.g., as a target cell or target particle) bound with a 4-μm diameter polystyrene bead (e.g., as a selection particle), the acoustophoretic mobility would have a positive value (about 31 fL), and migrate strongly to a pressure node (e.g., the axial center of the central channel(s) 220A or 220B in FIG. 2) in the fluid when acoustically actuated. Furthering this example, upon binding an additional selection particle to the complex, such as a 4.2-μm diameter silicone oil droplet with negative contrast, the net mobility would be very nearly zero and the three-particle complex would be unresponsive to acoustophoresis. This is similar to the bound particle 115C, which has relatively neutral acoustic contrast and binds to both the low-contrast selection particle 105A and the high-contrast selection particle 105B.

In addition, the same bound complex of T-cell with polystyrene bead could be made to have the inverse mobility of −31 fL by instead adding a larger silicone droplet with a diameter of 5.3 μm. In that case, the three-particle complex would migrate strongly to a pressure anti-node in the fluid when acoustically actuated. For example, if the acoustophoresis device (e.g., the first stage 205A shown in FIG. 2, other acoustophoresis stages described herein, etc.) is manufactured such that the pressure node is along the central longitudinal axis of the acoustic channel, and the pressure anti-node leads to the side channels at the outlet of the acoustic channel, the three-particle complex would be guided to the side channels of the acoustophoresis device. By leveraging these differences in acoustic contrast through the introduction of corresponding selection particles, precise and high-throughput separation of cells and other target particles can be achieved using acoustophoresis.

The above examples are illustrations and many other variations can be achieved. For example, rather than one single polystyrene particle of several microns, in some implementations smaller particles in larger quantity that would bind to the target cell at multiple locations. The selection particles described herein may have any suitable size, for example, ranging from 0.2 microns to 1 micron, from 1 micron to 4 microns, from 4 microns to 10 microns, or anywhere from 0.2 microns to 30 microns, among other ranges. A polystyrene bead of 1 micron diameter in quantity of 50 bound to the cell would have a mobility of about 28 fL, and this could be neutralized to zero by binding 50 additional 1.1 micron silicone oil droplets. The number of particles binding is determined by the binding kinetics of the affinity molecules (such as antibody) and the number of available binding sites on the cell surface.

Additionally, because the acoustic contrast of a cell or particle depends on the density and compressibility of the suspending fluid or media, other configurations (e.g., different selection particles, etc.) can be utilized for fluids other than saline buffer. For example, a T-cell can have contrast near zero in a solution of saline mixed with iodixanol to a density of about 1.1 g/ml². Similarly, adjusting the concentration of a density medium (e.g., any media having a density different from the suspension media, etc.) added to the suspending fluid can be used to alter the net contrast factor of a complex of multiple particles. For example, a three-particle complex of T-cell, polystyrene particle, and low-contrast paraffin particle could be suspended in a solution with density medium adjusted such that the paraffin particle has zero (e.g., neutral) contrast, while the T-cell and polystyrene particle have positive contrast and mobility. The net mobility would be positive. A first stage of separation (e.g., a first separation stage such as the stage 205A in FIG. 2) could be performed and then in a second stage (e.g., the second stage 205B in FIG. 2), iodixanol could be added such that the paraffin particle then has negative contrast, although the polystyrene has positive contrast, and the net mobility of the complex would become negative. An example of such a two-stage process is described herein in connection with FIG. 3.

The description of acoustophoretic mobility here is for illustrative purposes, and it should be understood that other descriptions of acoustophoretic mobility can be used to select the properties of the selection particles. For example, acoustophoretic mobility may instead be calculated to additionally account for the differences in drag force experienced by the particle or particle complex due to changes in volume V of the particles.

Various density mediums can be utilized with the techniques described herein: Some examples are iodixanol (e.g., Optiprep, etc.), polysaccharide (e.g., Ficoll, Histopaque, etc.), colloidal suspension (e.g., Percoll, etc.), dextran, polyethylene glycol, cesium chloride, and hetastarch. Some examples of selection particles can include hydrogels, gas bubbles, oil-filled vesicles, and the selection particles may incorporate nano-particles to tune the properties where the nanoparticles may include iron oxide, silica, cured silicone, gold, silver, carbon, etc.

Referring again to FIG. 2, as shown in the stage 205A, the outlet 210A feeds its output into the inlet of the second stage 205B. The second stage 205B can be its own acoustophoresis device, and may be separate from (e.g., on a different chip, etc.) the first stage of the acoustophoresis process. In some implementations, the second stage 205B can form a part of the same acoustophoresis device as the first stage 205A (e.g., part of the same chip, etc.). Any number of microfluidic features can exist between the first stage 205A and the second stage 205B, such as one or more pumps, valves, reservoirs, fluid capacitors, or other types of microfluidic features or devices. As shown, because the bound particles 115B were focused toward the central axial outlet 210B of the first stage 205A, the bound particles 115B are not received by the inlet of the second stage 205B. The second stage 205B of the example acoustophoresis system shown in FIG. 2 can be similar to the first stage 205A described herein above. The second acoustophoresis stage 205B having an inlet 215B, a central channel 220B, and multiple outlet channels 210D, 210E, and 210F. The central channel 220B of the stage 205B can be a microchannel fabricated from a substrate, such as silicon, glass or quartz, or a polymer with high acoustic impedance, such as polystyrene. The microchannel can be fabricated using any suitable technique, including but not limited to molding (e.g., injection molding), etching, embossing, laser ablation, or combinations thereof. The central channel 220B can be rectangular in cross section, with width and height dimensions that can range from 100 μm to 1000 μm. However, it should be understood that other sizes are possible to achieve desired outcomes, including, for example, 0 μm to 5 μm, 10 μm to 100 μm, or 1000 μm to 10000 μm. The central channel 220B need not necessarily be rectangular in cross-section, and may include any type of suitable geometry, such as a curved geometry, an elliptical geometry, a circular geometry, a hexagonal geometry, or an octagonal geometry, among others.

The inlet 215B of the second stage 205A can receive a suspension media provided as output (e.g., the outlet 210A) from a first stage of the acoustophoresis system depicted in FIG. 2. As shown in this example, the outlet 210A of the first stage 205A is provided as input to the inlet of the second stage 205B. Although not shown in FIG. 2A, it should be understood that any number of microfluidic features or devices (e.g., reservoirs, tubing, pumps, fluid capacitors, valves, etc.) may be present between the outlet 210A of the first stage 205A and the inlet 215B of the second stage 205B. In some implementations, the second stage 205B may be one of many parallel acoustophoresis channels, for example, forming a system that provides increased throughput of separated cells. Likewise, any number of outlets may feed into any number of inlets of one or more microfluidic stages, to achieve desired results based on the techniques described herein.

The outlets 210D, 210E, and 210F of the stage 205B can provide streams of the suspension media including target cells or target particles following an acoustophoresis process. In general, each of the outlets 210D, 210E, and 210F can align (e.g., along the length of the acoustophoresis channel) with a respective pressure node or pressure anti-node induced by a transducer in the suspension fluid during the operation of the second stage 205B. The outlets 210D, 210E, and 210F of the second stage 205B can be outlet ports, which can be connected to (e.g., via any type of connector or fastener, etc.) a pipe, a tube, a reservoir, a pump, or another microfluidic feature that can receive an output flow of the separated suspension fluid. Although three outlets 210D, 210E, and 210F of the second stage 205B are shown, it should be understood that any number of outlets may be implemented using the techniques described herein, for example, such that each outlet aligns with a respective pressure node or anti-node induced in the suspension media by a transducer. In some implementations, any of the outlets of any stage of the acoustophoresis device can be connected (e.g., via one or more connectors, tubes, pipes, etc.) to other microfluidic processing stages or other microfluidic features, as described herein.

As shown in the first stage 205A of the example system depicted in FIG. 2, the bound cells 115A and 115C are separated from the bound cells 115B. This can be considered a binary “NOT” operation, in which target cells having a B marker, but not the A marker, are isolated from other cells in the solution. This can be accomplished by leveraging the acoustic contrast of the selection particles bound to the cells 110A, 110B, and 110C. In this example, the only cell that includes a B marker, but does not include an A marker, the bound cell 115B, is separated from other cells in the solution. As such, two output streams are provided from the outlets 210A and 210B, the first being a solution including the bound cells 115A and 115C (both of which include an A marker), and a solution including the bound cells 115B. Separation is achieved in this stage due to the differences in acoustic contrast between the bound cells 115A, 115B, and 115C.

Recall that particles having a larger acoustic contrast can experience a greater force in response to actuation by a transducer during acoustophoresis, and therefore will migrate toward pressure nodes (e.g., along a central axis, etc.) of the acoustophoresis channel. As described herein, the bound particle 115B is bound to a high-contrast selection particle 105B, and therefore has a positive acoustic contrast relative to the bound particles 115A and 115C. Likewise, because the bound particles 115A and 115C have an acoustic contrast that is lower (e.g., negative or neutral) than the bound particle 115B, and would therefore migrate very little or would migrate toward an antinode (e.g., aligned with the side channel outlet 210A).

Then, in the second stage 205B, the bound cells 115A are separated from the bound cells 115B. This can be considered separation based on a binary “AND” operation, in which target cells having both the A and B markers are isolated from other cells having just the A marker. This can be accomplished by leveraging the acoustic contrast of the selection particles bound to the cells 110A and 110C, for example, relative to the media in which the bound particles 115A and 115C are suspended. In this example, the only cell that includes both an A marker and a B marker is the bound cell 115C, which migrates toward the central outlet 210E and is separated from the bound cells 115A. Similar to the first stage 205A, two output streams are provided from the outlets 210D and 210E, the first being a solution including the bound cell 115A (which only includes an A marker), and a solution including the bound cells 115C, which includes both the A and the B markers. Separation is achieved in this stage due to the differences in acoustic contrast between the bound cells 115A and 115C.

As described herein, the bound cells 115C have a neutral acoustic contrast relative to the other materials in the second stage 205B of the acoustophoresis system. Because the bound cells 115C have a larger acoustic contrast than the bound cells 115A (which, as described herein above, have a net negative acoustic contrast), the second stage 205B can be configured such that particles having a neutral acoustic contrast (e.g., the bound cells 115C) will not migrate and will remain flowing toward the central outlet 210E, while the cells having a negative acoustic contrast will migrate towards the side channel outlet 210D. This allows the bound cells 115A to be separated from both the bound cells 115B and the bound cells 115C across both stages 205A and 205B. Because acoustophoresis can be implemented as a high-throughput process (e.g., continuous flow from inlet channel to outlet and with a high concentration of cells in the suspension) and in parallel with other concurrent acoustophoresis devices, the systems and methods described herein provide a precise and high-throughput solution for target cell or target particle separation.

Referring now to FIG. 3, depicted is a diagram of another example multi-stage acoustophoresis separation system used to isolate target cell types, in accordance with one or more implementations. The system can be used, for example, in a two-stage selection process for two cell types. The system can be used, for example, to separate two desired cells from an unwanted cell type in a solution. As shown, the system has three stages: an incubation stage 300A, a first acoustophoresis stage 300B, and a second acoustophoresis stage 300C. The incubation phase can be similar to the incubation process described herein above in connection with FIG. 1B. The legend 330 of FIG. 3 provides three example cell types: a first target cell 305A, which has a marker that enables binding to the low-contrast particle 310A, a second target cell 305B, which has a marker that enables binding to the high-contrast particle 310B, and the unwanted cell 310C, which does not bind to the selection particles 310A or 310B.

The target cells 305A and 305B can be similar to the target cells 110A and 110B, and each of the target cells 305A and 305B can express surface markers that bind to the selection particles 310A and 310B, respectively. The unwanted cell 305C lacks the expression of such surface markers, and therefore will not bind to the selection particles 310A or 310B. The low-contrast particle 310A can be any type of low-contrast particle described herein, and can be similar to the low-contrast particle 105A described herein in connection with FIG. 1A. The high-contrast particle 310B can be any type of high-contrast particle described herein, and can be similar to the high-contrast particle 105B described herein in connection with FIG. 1A.

At stage 300A, the low-contrast particles 310A and the high-contrast particles 310B can be introduced into a solution 325 containing the target cells 305A and 305B, and the unwanted cell 305C. The solution 325 may be any type of suitable solution (e.g., an incubation media), such as saline, for example. The solution 325 can be stored in a container in which incubation takes place. In some implementations, the solution 325 in the container can be part of an acoustophoresis device, and can be fluidly coupled to an inlet of a stage of the acoustophoresis device (e.g., the first stage 300B). Because the target cell 305A includes a marker that binds the low-contrast selection particles 310A, the low-contrast selection particles 310A bind to the target cell 305A in solution, changing the net acoustic contrast of the target cell 305A. Likewise, because the target cell 305B includes a marker that binds the high-contrast selection particles 310B, the high-contrast selection particles 310B bind to the target cell 305B in the solution, changing the net acoustic contrast of the target cell 305A. The unwanted cell 305C does not bind to any of the selection particles 310A or 310B.

Once the target cells 305A and 305B have bound to the selection particles 310A and 310B (e.g., after a predetermined amount of time in solution, etc.), the solution including the bound target cells 305A and 305B, and the unwanted cells 305C, is introduced into the first stage 300B of the acoustophoresis system. As shown, the first stage 300B of the acoustophoresis system includes three inlet channels, which are fluidly coupled to a central channel (the main acoustophoresis channel), which itself is fluidly coupled to three outlet channels. Although three inlet and outlet channels are pictured here in the first stage 300B, it should be understood that any number of inlet and outlet channels are possible. The central channel of the first stage 300B can be a microchannel fabricated from a substrate, such as silicon, glass or quartz, or a polymer with high acoustic impedance, such as polystyrene. The microchannel can be fabricated using any suitable technique, including but not limited to molding (e.g., injection molding), etching, embossing, laser ablation, or combinations thereof. The central channel can be rectangular in cross section, with width and height dimensions that can range from 100 μm to 1000 μm. However, it should be understood that other sizes are possible to achieve a desired outcome, including, for example, 0 μm to 5 μm, 10 μm to 100 μm, or 1000 μm to 10000 μm. The central channel need not necessarily be rectangular in cross-section, and may include any type of suitable geometry, such as a curved geometry, an elliptical geometry, a circular geometry, a hexagonal geometry, or an octagonal geometry, among others.

The two side inlets of the first stage 300B can receive the solution including the bound target cells 310A and 310B, and the unwanted cells 310C. The solution can be, for example, saline, or another suitable fluid that can allow binding of the selection particles 310A and 310B to the target cells 305A and 305B, respectively. The inlets of the first stage 300B can be inlet ports, which can be connected to a pipe, a tube, a reservoir, a pump, or another microfluidic feature that provides the solution include the target cells 305A and 305B, and the unwanted cells 305C. In some implementations, the first stage 300B may be one of many parallel acoustophoresis channels, for example, to form a system that provides increased throughput of separated cells. In addition, the center inlet channel can receive an additional separation solution 315.

The separation solution 315 can be a solution selected to modify the density and compressibility of the suspending fluid within the first stage 300B. Some examples of density altering media can include iodixanol (Optiprep), Ficoll (polysaccharide), Histopaque, Percoll, dextran, polyethylene glycol, and cesium chloride, and hetastarch. In some implementations, the separation solution 315 can be the same as, or have similar density and compressibility as, the solution with which the cells 305A, 305B, and 305C are provided. The separation solution 315 may mix with the solution carrying the cells 305A, 305B, and 305C to alter the density and compressibility of the suspension fluid within the first stage 300B. In some implementations, the separation solution 315 may flow in laminar flow adjacent to the flow of the solution carrying the cells and serve to position cells near the sidewalls of the channel as they enter the acoustophoresis channel in the first stage 300B.

The outlets of the first stage 300B can provide streams of the suspension media including target cells or target particles following an acoustophoresis process. In general, each of the outlets can align (e.g., along the length of the acoustophoresis channel) with a respective pressure node or pressure anti-node induced by a transducer in the suspension fluid during the operation of the first acoustophoresis stage 300B. The outlets of the first stage 300B can be outlet ports, which can be connected to (e.g., via any type of connector or fastener, etc.) a pipe, a tube, a reservoir, a pump, or another microfluidic feature that can receive an output flow of the separated suspension fluid. Although three outlets are shown, it should be understood that any number of outlets may be implemented using the techniques described herein, for example, such that each outlet aligns with a respective pressure node or anti-node induced in the suspension media by a transducer. In some implementations, any one of the outlets an acoustophoresis device can be connected (e.g., via one or more connectors, tubes, pipes, etc.) to other microfluidic processing stages or other microfluidic features, as described herein.

As shown in the stage 300B, the unwanted cells 305C have an acoustic contrast that causes the unwanted cells 305C to not migrate significantly, or to migrate towards the side channel outlets of the stage 300B. The side channels can lead, for example, to a waste cell container or processing stage that removes, disposes of, or otherwise processes the unwanted cells 305C. Because the unwanted cells 305C are untreated with any type of particle, their acoustic contrast can differ from the acoustic contrast of the target cells 305A and 305B. In addition, the parameters (e.g., width, height, transducer frequency, separation solution 315 density and compressibility, etc.) of the stage 300B can be selected such that a pressure nodes or anti-nodes (depending on the implementation) guides the unwanted cells 305C toward the side channel outlets, and the target cells 305A and 305B toward the central channel outlet. As shown, the central channel outlet can lead to the inlet of the second stage 300C.

The second stage 300C can be structured similarly to the first stage 300B, but can instead include a single inlet that receives the output of the central outlet of the first stage 300B. As shown, the density medium 320 can be added to the output of the first stage 300B. The density medium can alter the density and compressibility of the fluid in the second stage 300C, thereby altering the response of the target cells 305A and 305B as described herein. The density medium 320 can be selected based on the properties of the cells 305A and 305B. Some examples of density media 320 include iodixanol (Optiprep), Ficoll (polysaccharide), Histopaque, Percoll, dextran, polyethylene glycol, and cesium chloride, and hetastarch, among others. As described herein, the central outlet of the first stage 300B may first lead to other microfluidic devices or features prior to the inlet of the second stage 300C. For example, the central outlet of the first stage 300B may be fluidly coupled to a reservoir or another type of mixing device, to which the density medium 320 is introduced to alter the solution's density and compressibility prior to introducing the solution to the second stage 300C.

The second stage 300C can include an inlet channel, which is fluidly coupled to a central channel (the main acoustophoresis channel), which itself is fluidly coupled to three outlet channels. Although one inlet and three outlet channels are pictured here in the second stage 300C, it should be understood that any number of inlet and outlet channels are possible. The central channel of the second stage 300C can be a microchannel fabricated from a substrate, such as silicon, glass or quartz, or a polymer with high acoustic impedance, such as polystyrene. The central channel can be rectangular in cross section, with width and height dimensions that can range from 100 μm to 1000 μm. However, it should be understood that other sizes are possible to achieve a desired outcome. As described herein, the dimensions of acoustophoresis microchannels can be chosen to achieve pressure nodes and anti-nodes at desired positions across the microchannel. The transducer(s) that generate the nodes or anti-nodes in the solution of the first stage 300B or the second stage 300C can operate at a predetermined frequency, which may be selected based on the dimension of the first stage 300B or the second stage 300C. The transducer can be controlled to carry out the acoustophoresis processes described herein.

The inlet of the second stage 300C can receive the solution including the bound target cells 310A and 310B, including the density media 320, with the unwanted cells 310C being previously separated from the solution in the first stage 300B. The inlet of the second stage 300C can be an inlet port, which can be connected to or coupled to a pipe, a tube, a reservoir, a pump, or another microfluidic feature that provides the solution include the target cells 305A and 305B in combination with the density media 320. In some implementations, the second stage 300C may be one of many parallel acoustophoresis channels, for example, to form a system that provides increased throughput of separated cells. In some implementations, the second stage 300C can form a part of the first stage 300B (e.g., part of the same acoustophoresis chip, etc.).

The outlets of the second stage 300C can provide a central output stream including the target cells 305B (which are bound to the high-contrast particles 310B) and two side channel output streams including the target cells 305A (which are bound to the low-contrast particles 310A). As described herein, each of the outlets can align (e.g., along the length of the acoustophoresis channel) with a respective pressure node or pressure anti-node induced by a transducer in the suspension fluid during the operation of the second stage 300C. The outlets of the second stage 300C be outlet ports, which can be connected to (e.g., via any type of connector or fastener, etc.) a pipe, a tube, a reservoir, a pump, or another microfluidic feature that can receive an output flow of the separated suspension fluid. In some implementations, any one of the outlets of the second stage 300C can be connected (e.g., via one or more connectors, tubes, pipes, etc.) to other microfluidic processing stages or other microfluidic features, as described herein.

The operation of the second stage 300C is similar to that of the first stage 300B, but the forces experienced by each of the target cells 305A and 305B are different due to the introduction of the density media 320. Because the density media 320 changes the density and compressibility of the solution, the relative net acoustic contrast of each of the target cells 305A and 305B are also changed. In this example, the high-contrast cells 305B (e.g., bound to the high-contrast selection particles 310B) migrate toward the central outlet in response to actuation by the transducer coupled to the second stage 300C. At the same time, the low-contrast cells 305A (e.g., bound to the low-contrast selection particles 310A) migrate toward the side channel outlets of the second stage 300C. As described herein, the second stage 300C can be a part of a number of acoustophoresis stages operating in parallel, thereby providing overall improved throughput over other implementations.

Although the acoustophoresis devices shown in FIGS. 2 and 3 are continuous-flow acoustophoresis devices, it should be understood that the techniques described herein are compatible with any type of acoustic device. For example, the techniques described herein may be implemented similarly in connection with acoustic trapping devices, surface acoustic wave devices, macroscale acoustic separators, or combinations thereof. Because the techniques described herein operate via manipulation of the acoustic contrast of target particles, any device that uses acoustic contrast to manipulate cells particles is compatible with and may benefit from the techniques described herein.

Although the acoustophoresis devices shown in FIGS. 2 and 3 show a small number of particles in solution, it should be understood that the techniques described herein are compatible with high concentrations of target particles in the suspension, and many particles may occupy the microchannel and outlets at the same time. For example the concentration of target particles in suspension may be between 10³/ml and 10¹⁰/ml, 10⁴/ml and 10⁸/ml, or 10⁵/ml and 10⁷/ml.

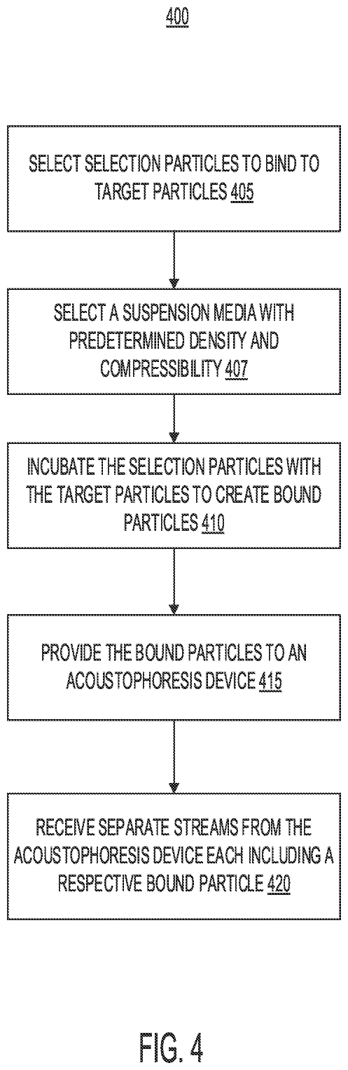

Referring now to FIG. 4, illustrated is a flow diagram of an example method 400 of selective separation of target cells using selection particles in acoustophoresis, in accordance with one or more implementations. In brief overview, the method 400 can include selecting a set of selection particles (e.g., one or more of the particles 105A, 105B, 310A, or 310B, any other selection particles described herein, etc.) to bind to target particles (e.g., the target cells 110A, 110B, 305A, or 305B, any other target cells or target particles as described herein, etc.) for use in acoustophoresis (STEP 405), incubating the set of selection particles with the target particles to create bound particles (STEP 410), providing the bound particles as input to an acoustophoresis device (STEP 415), and receiving separate streams that each include a respective one of the target particles (STEP 420).

In further detail, the method 400 can include selecting a set of selection particles (e.g., one or more of the particles 105A, 105B, 310A, or 310B, any other selection particles described herein, etc.) to bind to target particles (e.g., the target cells 110A, 110B, 305A, or 305B, any other target cells or target particles as described herein, etc.) for use in acoustophoresis (STEP 405). As described herein, certain selection particles can bind to surface markers on target particles to alter the net acoustic contrast of those particles when provided to an acoustophoresis device. The target particles can be any type of target particle or target cell. For example, a target particle can be a target cell, with certain surface markers (e.g., proteins) expressed on the surface of its cell membrane. In some implementations, a target particle can be any type of particle having a feature to which a selection particle can bind.

Some examples of selection particles can include, but are not limited to, gas-filled particles, oil-filled particles, or porous particles, such that they have low density and high compressibility. By having such properties, these selection particles can result in low acoustic contrast (e.g., when suspended in a separation medium, etc.). In some implementations, some example selection particles can include high-density materials, such as nano-particles of gold, iron-oxide, or other high-density materials. Such selection particles can have a high density and low compressibility, resulting in high acoustic contrast (e.g., when suspended in a separation medium, etc.). A selection particle can be selected based on the markers expressed on the surface a target particle or cell, or based on a desired change in net acoustic contrast for each target (or in some implementations, undesired) particle or cell. In some implementations, a set of selection particles can include selecting one type of selection particle, while in some implementations, many selection particles may be chosen to bind to a larger number of surface marker sites on one or more target particles. Selecting one or more selection particles can be based upon the markers (or lack thereof) that can be present on one or more target particles or undesired particles, as described herein.

One or more selection particles can be selected so as to bind to one target particle, and not to bind to a second particle. Two or more selection particles can be selected such that any number of the selection particles bind to a first target particle, and a different number of selection particles bind to a second particle. It will be appreciated that any number of selection particles may be selected for solutions including any number of target particles or undesired particles. Undesired particles may also be considered target particles, and it should be understood that the term “undesired” is provided merely as an example to indicate that, in the particular example, said particles may be disposed of or unused in further processing steps.

The method 400 can include selecting a suspension media having a predetermined densify and compressibility (STEP 407). The suspension media can be selected to achieve a desired acoustic contrast for the complex of the target particles when bound to the one or more selection particles. An example of a selected suspension media can include an isotonic buffer, such as phosphate buffered saline or a cell culture medium. The isotonic buffer may have a density ranging from 1.005 g/mL to 1.01 g/mL, and may have a compressibility of around 4.4*10⁻¹⁰ Pa⁻¹. Other types of suspension media may also be selected, such as media having a density up to about 1.4 g/mL and with a compressibility of about 3.2*10⁻¹⁰ Pa⁻¹, or media having a density of about 0.95 g/mL and a compressibility of about 5*10⁻¹⁰ Pa⁻¹, or media having a density of anywhere between 0.95 g/mL and 1.4 g/mL and a compressibility between 3.2*10⁻¹⁰ Pa⁻¹ and 5*10⁻¹⁰ Pa⁻¹.

In an embodiment, selecting the suspension media may include selecting a fluid additive with a predetermined density and compressibility. The fluid additive may be a density media that has characteristics that, when added to the suspension media, cause the density and compressibility of the suspension media change to predetermined values. The fluid additive may be added at any point in the method 400, including prior to introduction to an acoustophoresis channel, or between acoustophoresis stages as described herein. In an embodiment, the fluid additive may include a growth media, or may include additional particles.

The method 400 can include incubating the set of selection particles with the target particles to create bound particles (STEP 410). Once the selection particles and the suspension media have been chosen for the acoustophoresis process, the selection particles can be incubated, or introduced into the same solution (e.g., the selected suspension media or in a separate incubation media) as the target particles or cells. The target particles or cells can be provided in a solution, such as a saline solution or other solutions (e.g., which may include the various density media described herein, etc.). In the case of cells, where the surface markers are particular proteins expressed on the surface of the cell membranes, the target particles may bind with a particular protein on the target cell during incubation. Binding selection particles to the target particles can be performed using a variety of bonding techniques or affinity molecules. For example, selection particles may bind to target particles using any of antibodies, aptamers, antigen-binding fragments, lectins, or other biochemistry-related molecules that allow a selection particle to bind to a surface or portion of a target particle. Examples of selection particles binding target particles are described in greater detail in connection with FIGS. 1A, 1B, and 3. In some implementations, the target cells and the selection particles can be suspended in a solution that facilitates binding of the selection particles to the target particles. Once the selection particles have bound to the surface of the target particles in solution to alter the net acoustic contrast of the target particles (e.g., creating “bound particles”), the method 400 can proceed to STEP 415.

The method 400 can include providing the bound particles in the selected suspension media as input to an acoustophoresis device (STEP 415). Once the bound particles are created by binding the selection particles to the surface of the target particles, the solution including the bound particles can be provided as input to an acoustophoresis device, such as the acoustophoresis devices shown in FIGS. 2 and 3. Providing the bound particles can include flowing the particles through a channel of the acoustophoresis device, as described herein. In some implementations, the acoustophoresis device can be a multi-stage acoustophoresis device, such as the acoustophoresis systems described herein in connection with FIGS. 2 and 3. In some implementations, providing the bound particles as input to an acoustophoresis device can include introducing a density media to the solution including the bound particles. Doing so can alter the density and compressibility of the solution including the bound particles, and thereby alter the relative force experienced by each bound particle when exposed to acoustic energy. Different forces can be experienced by a bound particle depending on whether its net acoustic contrast is positive, negative, or neutral. In implementations where an acoustophoresis device includes multiple stages, in some implementations, additional or different density media can be introduced to the solution including the bound particles to alter their response to acoustophoresis in the upcoming stage.

The method 400 can include receiving separate streams that each include a respective one of the target particles (STEP 420). By providing the target particles, which are treated with and bound to selection particles, to an acoustophoresis system tuned to separate the bound particles according to their acoustic contrast, precise and high-throughput cell separation can be achieved. In doing so, multiple output streams are produced that each include at least types of the target particles (e.g., bound to a selection particle) separated from the other types of target particles in the original solution. In some implementations, the separate streams can be provided across multiple stages of the acoustophoresis device. For example, in FIG. 2, the target particles 115B are provided as output of the first stage, while in the second stage, separation of the target particles 115A from the target particles 115C occurs. In some implementations, unwanted particles (which may or may not be bound to one or more selection particles) can be received in one or more fluid streams at one or more outputs of the acoustophoresis device. For example, as shown in in FIG. 3, the unwanted cells 305C are provided at the output of the side channels of the first stage 300B.

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only as well as both ‘A’ and

Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements.

The devices, systems, and methods described herein may be embodied in other specific forms without departing from the characteristics thereof. The foregoing implementations are illustrative rather than limiting of the described devices, systems, and methods. Scope of the devices, systems, and methods described herein is thus indicated by the appended claims, rather than the foregoing description, and changes that come within the meaning and range of equivalency of the claims are embraced therein.

What is claimed is:

1. A method of acoustophoresis using selection particles that alter acoustic response of particles in suspension, comprising:
selecting a plurality of selection particles based on a first marker of a first target particle and a second marker of a second target particle, the plurality of selection particles comprising a first selection particle having a greater acoustic contrast and a second selection particle having a lesser acoustic contrast;
incubating the plurality of selection particles in a solution comprising at least the first target particle and the second target particle, such that the first selection particle binds to the first marker of the first target particle and the second selection particle binds to the second marker of the second target particle;
providing the solution to an acoustophoresis channel configured to separate the first target particle from the second target particle; and
receiving a first output stream comprising the first target particle bound to the first selection particle and a second output stream comprising the second target particle bound to the second selection particle from the acoustophoresis channel.

2. The method of claim 1, wherein the first target particle and the second target particle comprise cells, and the first marker and the second marker are expressed on a surface of a cell membrane of each of the first target particle and the second target particle, respectively.

3. The method of claim 2, wherein the first marker and the second marker comprise a protein.

4. The method of claim 1, wherein the solution comprises three or more target particles, the three or more target particles including the first target particle and the second target particle.

5. The method of claim 1, wherein the solution is provided to the acoustophoresis channel after the plurality of selection particles have incubated in the solution for a predetermined amount of time.

6. The method of claim 1, further comprising controlling a transducer connected to the acoustophoresis channel to carry out acoustophoresis.

7. The method of claim 1, further comprising providing a fluid additive having a predetermined density and compressibility with the solution to the acoustophoresis channel to modify an acoustic contrast of the first target particle and the second target particle in the solution including the fluid additive.

8. The method of claim 1, wherein providing the solution to the acoustophoresis channel comprises flowing the solution through the acoustophoresis channel.

9. The method of claim 1, wherein the acoustophoresis channel comprises a first outlet that provides the first output stream and a second outlet that provides the second output stream.

10. The method of claim 1, wherein the solution further comprises a third particle, and wherein the first output stream received from the acoustophoresis channel comprises the third particle.

11. The method of claim 10, further comprising providing the first output stream and a fluid additive to a second acoustophoresis channel configured to separate the first target particle from the third particle based on a net acoustic contrast of a complex of the first target particle and the first selection particle and an acoustic contrast of the third particle, in the second acoustophoresis channel.

12. The method of claim 11, further comprising receiving a third output stream comprising the first target particle and a fourth output stream comprising the third particle.

13. A method of acoustophoresis using selection particles that have predetermined size, density, and compressibility, comprising:
selecting a first selection particle and a second selection particle based on a first marker and a second marker of a first target particle and based on a second target particle, the first selection particle having a high acoustic contrast when suspended in a selected fluid;
incubating the first selection particle and the second selection particle in a solution comprising the selected fluid, the first target particle, and the second target particle, such that the first selection particle binds to the first marker of the first target particle and the second selection particle binds to the second marker of the first target particle;
providing the solution to an acoustophoresis channel configured to separate the first target particle from the second target particle based on a difference between a net acoustic contrast of a complex of the first selection particle, the second selection particle, and the first target particle and an acoustic contrast of the second target particle; and
receiving a first output stream comprising the first target particle bound to the first selection particle and the second selection particle and a second output stream comprising the second target particle from the acoustophoresis channel.

14. The method of claim 13, wherein the solution is provided to the acoustophoresis channel after the first and second selection particles have incubated in the solution for a predetermined amount of time.

15. The method of claim 13, further comprising providing a fluid additive having a predetermined density and compressibility with the solution to the acoustophoresis channel to modify an acoustic contrast of the complex of the first target particle bound to the first selection particle and the second selection particle, and an acoustic contrast of the second target particle.

16. The method of claim 13, further comprising controlling a transducer that causes the first target particle bound to the first selection particle and the second selection particle to be forced towards a center of the acoustophoresis channel.

17. A system, comprising:
an acoustophoresis device comprising a microfluidic channel and configured to:
receive a solution including a first target particle, a second target particle, a first selection particle, and a second selection particle, the solution having been incubated such that the first target particle is bound to the first selection particle and the second selection particle in the solution such that a net acoustic contrast of a complex of the first target particle, the first selection particle, and the second selection particle differs from an acoustic contrast of the second target particle;
perform acoustophoresis in the microfluidic channel; and
provide a first output stream comprising the first target particle bound to the first selection particle and the second selection particle, and a second output stream comprising the second target particle.

18. The system of claim 17, further comprising a container comprising incubation media including the first selection particle, the second selection particle, the first target particle, and the second target particle, such that the first selection particle and the second selection particle bind to the first target particle in the incubation media.

19. The system of claim 18, wherein the container is fluidly coupled to an inlet of the microfluidic channel of the acoustophoresis device, and the incubation media is provided at least as part of the solution to the microfluidic channel of the acoustophoresis device.

20. The system of claim 17, wherein the microfluidic channel is a first microfluidic channel, and the acoustophoresis device further comprises a second microfluidic channel that receives one of the first output stream or the second output stream and performs acoustophoresis.
